(19) United States
(12) Patent Application Publication
(10) Pub. No.: US 2006/0035818 A1
(43) Pub. Date: Feb. 16, 2006

(54) Use of corticotroph-derived glycoprotein hormone to induce lipolysis

(75) Inventors: James D. Kelly, Mercer Island, WA (US); Philippa J. Webster, Seattle, WA (US)

Correspondence Address: Robyn Adams;ZymoGenetics, Inc., Patent Department, 1201 Eastlake Avenue East, Seattle, WA, 98102, US

(73) Assignee: ZymoGenetics, Inc.

(21) Appl. No.: 11/253,881

(22) Filed: Oct. 19, 2005

Related U.S. Application Data
(63) Continuation of application No. 10/196,437, filed on Jul. 15, 2002, now abandoned.
(60) Provisional application No. 60/305,284, filed on Jul. 13, 2001.

(51) Int. Cl. A61K 38/22

(52) U.S. Cl. 514/8

(57) ABSTRACT

The use of corticotroph-derived glycoprotein hormone (CGH) to induce lipolysis, treat obesity, insulin resistance, and type II diabetes is described.

### REFERENCE TO RELATED APPLICATIONS

This application is a continuation of U.S. application Ser. No. 10/196,437, filed Jul. 15, 2002, herein incorporated by reference, which claims the benefit of U.S. Provisional Application Ser. No. 60/305,284, filed Jul. 13, 2001.

### FIELD OF THE INVENTION

The present invention relates to the treatment of obesity. More particularly, the invention relates to the use of corticotroph-derived glycoprotein hormone (CGH) to stimulate lipolysis for the treatment of obesity and diabetes.

### BACKGROUND OF THE INVENTION

The teachings of all of the references cited herein are incorporated in their entirety herein by reference.

Obesity is a public health problem, which is both serious and widespread. One third of the population in industrialized countries has an excess weight of at least 20% relative to the ideal weight. This phenomenon has spread to the developing world, particularly to the regions of the globe where economies are modernizing. As of the year 2000, there were an estimated 300 million obese people worldwide.

Obesity considerably increases the risk of developing cardiovascular or metabolic diseases. For an excess weight greater than 30%, the incidence of coronary diseases is doubled in subjects under 50 years of age. Studies carried out for other diseases are equally revealing. For an excess weight of 20%, the risk of high blood pressure is doubled. For an excess weight of 30%, the risk of developing non-insulin dependent diabetes is tripled, and the incidence of dyslipidemia increased six fold. The list of additional diseases promoted by obesity is long; abnormalities in hepatic function, digestive pathologies, certain cancers, and psychological disorders are prominent among them.

Treatments for obesity include restriction of caloric intake, and increased caloric expenditure through physical exercise. However, the treatment of obesity by dieting, although effective in the short-term, suffers from an extremely high rate of recidivism. Treatment with exercise has been shown to be relatively ineffective when applied in the absence of dieting. Other treatments include gastrointestinal surgery or agents that limit the absorption of dietary lipids. These strategies have been largely unsuccessful due to side-effects of their use.

Clearly there remains a need for novel treatments that are useful for reducing body weight in humans. Therapies that can be administered to promote lipolysis and weight loss would help to control obesity and thereby alleviate many of the negative consequences associated with this condition.

### DESCRIPTION OF THE INVENTION

Introduction

The present invention fills the need for a novel therapy to promote weight loss.

The present invention is comprised of administering corticotroph-derived glycoprotein hormone (CGH) to an individual to promote weight loss and in particular to promote lipolysis. The present invention is further comprised of a method for treating type-2 diabetes in an individual comprising administering a pharmaceutically effective amount of CGH to said individual. In another embodiment the present invention is comprised of a method for improving insulin sensitivity in an individual comprising administering a pharmaceutically effective amount of CGH to said individual.

Herein we disclose methods that are useful for the treatment of obesity. As described below, the ability to stimulate lipolysis in adipose tissue provides a means of intervening in a wide number of pathologies associated with obesity. In particular, we have discovered that CGH, when administered in vitro or in vivo, stimulates lipolysis. As a consequence, metabolic rate is increased, leading to decreased weight and increased insulin sensitivity.

When used to promote lipolysis, CGH can promote weight loss. The invented composition and methods are useful for treating conditions that include: obesity, atherosclerosis associated with obesity, diabetes, hypertension associated with obesity or diabetes, or more generally the various pathologies associated with obesity.

In another aspect of the invention, this agent can be used for the maintenance of weight loss in individuals treated with other medicaments that induce weight loss.

A preferred embodiment of the invention is the treatment of non-insulin dependent diabetes, especially that associated with obesity. In one embodiment, the use of CGH to treat non-insulin dependent diabetes is envisioned in non-obese individuals.

Yet another aspect of the invention relates to the use of CGH to increase resting metabolic rate in individuals. In one embodiment of this aspect, individuals with low resting metabolic rate are administered CGH to promote lipolysis and increase energy utilization.

Definitions and Terms

One aspect of the invention is the use of the novel glycoprotein hormone CGH to stimulate lipolysis. CGH is disclosed in International Patent Application No. PCT/US01/09999, publication no. WO 01/73034. It is comprised of an alpha subunit, glycoprotein hormone alpha2 (GPHA2), and a beta subunit, glycoprotein hormone beta (GPHB5). GPHA2 was previously called Zsig51 (International Patent Application No. PCT/US99/03104, publication no. WO 99/41377 published Aug. 19, 1999). SEQ ID NO: 1 is the human cDNA sequence that encodes the full-length polypeptide GPHA2, and SEQ ID NO:2 is the full-length polypeptide sequence of human GPHA2. SEQ ID NO:3 is the mature GPHA2 polypeptide sequence without the signal sequence. SEQ ID NO: 4 is the human cDNA sequence that encodes the full-length GPHB5 polypeptide. SEQ ID NO: 5 is the full-length GPHB5 polypeptide. SEQ ID NO: 6 is the mature GPHB5 polypeptide without the signal sequence. SEQ ID NO: 7 is the human genomic DNA sequence that encodes the full-length GPHB5 polypeptide.

The present invention relates generally to methods that are useful for stimulating lipolysis in adipose tissue. Those having ordinary skill in the art will understand that lipolysis is the biochemical process by which stored fats in the form of triglycerides are released from fat cells as individual free fatty acids into the circulation. Stimulation of lipolysis has been clearly linked to increased energy expenditure in humans, and several strategies to promote lipolysis and increase oxidation of lipids have been investigated to promote weight loss and treat the diabetic state associated with obesity. These therapeutic efforts primarily focus on creating compounds that stimulate the sympathetic nervous system (SNS) through its peripheral β-adrenoreceptors. The discovery of CGH-promoted lipolysis in adipose tissue presents a novel and specific method of treating obesity, and the insulin-resistant diabetic state associated with obesity.

As used herein, the terms “obesity” and “obesity-related” are used to refer to individuals having a body mass which is measurably greater than ideal for their height and frame. Preferably these terms refer to individuals with body mass index values of greater than 20, more preferably with body mass index values of greater than 30, and most preferably with body mass index greater than 40.

Overview

Energy expenditure represents one side of the energy balance equation. In order to maintain stable weight, energy expenditure should be in equilibrium with energy intake. Considerable efforts have been made to manipulate energy intake (i.e., diet and appetite) as a means of maintaining or losing weight; however, despite enormous sums of money devoted to these approaches, they have been largely unsuccessful. There have also been efforts to increase energy expenditure pharmacologically as a means of managing weight control and treating obesity. Increasing energy metabolism is an attractive therapeutic approach because it has the potential of allowing affected individuals to maintain food intake at normal levels. Further, there is evidence to support the view that increases in energy expenditure due to pharmacological means are not fully counteracted by corresponding increases in energy intake and appetite. See Bray, G. A. (1991) Annu Rev Med 42, 205-216.

Energy expenditure can be stimulated pharmacologically by manipulation of the central nervous system, by activation of the peripheral efferents of the SNS, or by increasing thyroid hormone levels. Much of the energy expended on a daily basis derives from resting metabolic rate (RMR), which comprises 50-80% of the total daily energy expenditure. For a review, see Astrup, A. (2000) Endocrine 13, 207-212. Noradrenaline turnover studies have shown that most of the variability in RMR unexplained by body size and composition is related to differences in SNS activity, suggesting that SNS activity does modulate RMR. See Snitker, S., et al. (2001) Obes. Rev. 1, 5-15. Meal ingestion is accompanied by increased SNS activity, and studies have demonstrated that increased SNS activity in response to a meal accounts for at least part of meal-induced thermogenesis.

The peripheral targets of the SNS involved in the regulation of energy utilization are the β-adrenoreceptors (β-AR's). These receptors are coupled to the second messenger cyclic adenosine monophosphate (cAMP). Elevation of cAMP levels leads to activation of protein kinase A (PKA), a multi-potent protein kinase and transcription factor eliciting diverse cellular effects. See Bourne, H. R., et al. (1991) Nature 349, 117-127. Adipose tissue is highly enervated by the SNS, and possesses three known subtypes of β-adrenoreceptors, β₁-, β₂-, and β₃-AR. Activation of the SNS stimulates energy expenditure via coupling of these receptors to lipolysis and fat oxidation. Increased serum free fatty acids (FFAs) produced by adipose tissue and released into the bloodstream stimulate energy expenditure and increase thermnogenesis. For a review, see Astrup, A. (2000) Endocrine 13, 207-212. In addition, elevated PKA levels increase energy utilization in fat by up-regulating uncoupling protein-1 (UCP-1), which creates a futile cycle in mitochondria, generating waste heat.

Over the past two decades, investigation of the physiological benefits of SNS activation for the treatment of obesity and diabetes related to obesity has centered on pharmacological activation of the β₃-AR. Expression of the β₃-AR is restricted to a narrower range of tissues than the β₁ or β₂ isoforms, and is highly expressed in rodent adipose tissue compared to the other isoforms. Experimental work in rodents treated with β₃-AR agonists has demonstrated that stimulation of lipolysis and fat oxidation produces increased energy expenditure, weight loss, and increased insulin sensitivity. See de Souza, C. J. and Burkey, B. F. (2001) Curr Pharm Des 7, 1433-1449. The potential benefits of these compounds have not been not realized, however, due to their lack of efficacy at the human β₃-AR. Further, it was only subsequently realized that the levels of β₃-AR in rodent adipose tissue are much higher than in human adipose tissue. In human adipose tissue, the β₁ and β₂ isoforms represent the predominant adrenoreceptor isoforms. See Arch, J. R. (2002) Eur J Pharmacol 440, 99-107. Thus, although the biochemical premise of stimulation of lipolysis for treatment of obesity has been clearly demonstrated, the mechanism for therapeutically producing the corresponding effects in humans is unrealized.

Strategies to promote lipid oxidation through lipolysis have demonstrated improved insulin sensitivity at doses that do not promote weight loss, and over time periods that do not affect body weight. It is not suprising that an insulin-sensitizing effect is more readily detectable than an anti-obesity effect. Stimulation of fat oxidation may rapidly lower the intracellular concentration of metabolites that modulate insulin signaling. The anti-obesity effect, by contrast, must develop gradually as large stores of fat are oxidized.

### BRIEF DESCRIPTION OF THE DRAWINGS

FIG. 1. Dose response of CGH and isoproterenol-induced lipolysis in 3T3 L1 adipocytes. Glycerol (panel A) and FFA (panel B) accumulations were determined following a 4-hour treatment with CGH (solid squares) or isoproterenol (solid triangles) at the indicated concentrations.

FIG. 2. Stimulation of lipolysis in vivo by CGH. Mice (n=4, each group) were injected IP with vehicle saline, CGH (300 μg/kg), or CL 316,243 (1 mg/kg). Changes in serum glycerol (upper panel, A) and FFA (lower panel, B) over a 2-hour period as described in Example 3 are shown for each group.

CGH Promotes Elevation of cAMP in Adipose Tissue

CGH exerts its effects through interaction with the thyrotropin-stimulating hormone (TSH) receptor. See Nakabayashi, K., et al. (2002) J Clin Invest 109, 1445-1452. The TSH receptor (TSHR) is a member of the G-protein coupled, seven transmembrane receptor superfamily. Activation of the TSH receptor leads to coupling with heterotrimeric G proteins, which evoke downstream cellular effects. The TSH receptor has been shown to interact with G proteins of subtypes Gₛ, G_{q}, G₁₂, and Gᵢ. In particular, interaction with Gₛ leads to activation of adenyl cyclase and increased levels of cAMP. See Laugwitz, K. L., et al. (1996) Proc Natl Acad Sci USA 93, 116-120.

Although the presence of TSH receptors in adipose tissue has been the subject of controversy for some time, recent reports have documented the presence of TSHR in adipose tissue of humans and rodents. Se, Bell, A., et al. (2000) Am J Physiol Cell Physiol 279, C335-340, and Endo, T., et al. (1995) J Biol Chem 270, 10833-10837.

Example 1 demonstrates the production of elevated cAMP by CGH in cultured murine 3T3-L1 adipocytes and in primary human adipocytes. We have discovered that CGH produces activation of a luciferase reporter gene construct under the control of cAMP response element (CRE) enhancer sequences. We typically observe a 15-40 fold induction of the luciferase reporter gene in response to CGH treatment, indicating significant production of cAMP in adipocytes following activation of the TSHR. These data suggest that CGH could be an important physiological regulator of adipose tissue lipolysis, which is primarily controlled by intracellular cAMP levels. For a review, see Astrup, A. (2000) Endocrine 13, 207-212.

CGH Promotes Lipolysis in Adipocytes and Whole Animals

CGH was examined for its ability to activate lipolysis in cultured 3T3-L1 murine adipocytes. Following treatment of adipocytes for 4 hours, lipolysis was assessed by the accumulation of glycerol and FFA in the adipocyte culture medium. Treatment of adipocytes with 10 nM human recombinant CGH produced significantly elevated levels of extracellular glycerol and FFA. Example 2 compares the lipolytic activity of CGH to isoproterenol, a non-specific β-adrenergic agonist. Maximal lipolysis achieved with CGH is at least 50% of that produced by isoproterenol. Lipolysis was significantly stimulated by CGH at concentrations of 0.1 nM, indicating that CGH is a potent regulator of lipolysis in adipocytes.

CGH also produced elevations in serum glycerol and FFA following IP injection into mice. As described in example 3, mice were fasted overnight before IP injection of either CGH (300 μg/kg), β3-AR agonist CL 316,243 (1 mg/kg), or vehicle saline. Serum was withdrawn before injection, or 2 hours post-injection. Although the vehicle controls showed decreases in serum glycerol and FFA levels, the animals treated with CGH showed significant elevations in both, indicating that CGH is a potent stimulator of lipolysis in vivo.

Advantages of CGH as a Lipolysis Stimulating Agent

CGH presents a novel method of producing lipolysis and increasing metabolic rate. Other strategies employed thus far have suffered from lack of specificity, such as β-AR agonists in general, or lack of efficacy, as for the most specific of the β₃-AR agonists developed thus far. Most of the agents investigated for human use have not exhibited sufficient selectivity and as a result, have produced increased blood pressure and heart rate due to activation of sympathetic pathways in tissues other than adipose. See Arch, J. R. (2002) Eur J Pharmacol 440, 99-107.

In spite of the emphasis on development of β₃-AR specific agonists, recent human studies have implicated the β₁- and β₂-adrenoreceptors as the primary mediators of sympathetically induced thermogenesis and energy expenditure. Further, studies in human obese populations suggest that decreases in resting metabolic rate observed in these individuals are the result of impaired function of β₂-adrenoreceptors in adipose tissue. See Schiffelers, S. L., et al. (2001) J Clin Endocrinol Metab 86, 2191-2199, and Blaak, E. E., et al. (1993) Am J Physiol 264, E11-17. Thus, a novel mechanism of increasing lipolysis without invoking sympathetic enervation presents a unique opportunity for the treatment of obesity.

Other studies in human lean and obese subjects have found that increases in plasma FFA levels lead to similar increases in lipid oxidation and energy expenditure. These studies conclude that the accumulation of fat in obese subjects may be due to a defect in adipose tissue lipolysis rather than to defects in lipid utilization. See Schiffelers, S. L., et al. (2001) Int J Obes Relat Metab Disord 25, 33-38.

Increased adipose lipolysis and the resulting decrease in adipocyte size are negatively correlated with insulin resistance in human cross-sectional studies. See Weyer, C., et al. (2000) Diabetologia 43, 1498-1506. Thus a method for stimulating lipolysis and reducing adipocyte size is predicted to decrease the insulin-resistant diabetic state associated with obesity. The presence of significant numbers of CGH receptors in adipose tissue represents a novel method for the control of lipolysis and RMR in human obese populations.

Use of CGH to Treat Type-2 Diabetes

CGH can also be administered to treat type-2 diabetes mellitus (Type II DM). Type II DM is usually the type of diabetes that is diagnosed in patients older than 30 years of age, but it also occurs in children and adolescents. It is characterized clinically by hyperglycemia and insulin resistance. Type II DM is commonly associated with obesity, especially of the upper body (visceral/abdominal), and often occurs after weight gain.

Type II DM is a heterogeneous group of disorders in which hyperglycemia results from both an impaired insulin secretory response to glucose and a decreased insulin effectiveness in stimulating glucose uptake by skeletal muscle and in restraining hepatic glucose production (insulin resistance). The resulting hyperglycemia may lead to other common conditions, such as obesity, hypertension, hyperlipidemia, and coronary artery disease.

CGH can be administered to an individual at dosages described below. COH can also be administered in conjunction with insulin, and other diabetic drugs such as tolbutamide, chlorpropamide, acetohexamide, tolazamide, glyburide, glipizide, glimepiride, metformin, acarbose, troglitazone and repaglinide.

Formulations and Administration of CGH

CGH can be administered to a human patient, alone or in pharmaceutical compositions where it is mixed with suitable carriers or excipient(s) at therapeutically effective doeses to treat or ameliorate diseases associated with obesity and diabetes. Treatment dosages of CGH should be titrated to optimize safety and efficacy. Methods for administration include intravenous, intraperitoneal, rectal, intranasal, subcutaneous, and intramuscular. Pharmaceutically acceptable carriers will include water, saline, and buffers, to name just a few. Dosage ranges would ordinarily be expected from 0.1 μg to 0.1 mg per kilogram of body weight per day. A useful dose to try initially would be 25 μg/kg per day. However, the doses may be higher or lower as can be determined by a medical doctor with ordinary skill in the art. For a complete discussion of drug formulations and dosage ranges see Remington's Pharmaceutical Sciences, 17^{th} Ed., (Mack Publishing Co., Easton, Pa., 1990), and Goodman and Gilman's: The Pharmacological Basis of Therapeutics, 9^{th} Ed. (Pergamon Press 1996).

### EXAMPLE 1

### CGH Activation of 3T3 L1 Adipocytes and Human Adipocytes Results in cAMP Production

Summary

Differentiated murine 3T3 L1 adipocytes and primary human adipocytes were used to study signal transduction of CGH. 3T3 L1 fibroblasts were differentiated into adipocytes and the cells were transduced with recombinant adenovirus containing a reporter construct, a firefly luciferase gene under the control of cAMP response element (CRE) enhancer sequences. This assay system detects cAMP-mediated gene induction downstream of activation of Gₛ-coupled G-protein coupled receptors (GPCR's). Treatment of the differentiated 3T3 L1 cells with isoproterenol, a β-adrenoreceptor agonist, resulted in elevation of cAMP levels and an 80-fold induction of luciferase expression. Treatment of differentiated 3T3 L1 cells with CGH also resulted in elevated cAMP levels and a 27-fold induction of luciferase expression. In a separate experiment, undifferentiated 3T3 L1 fibroblasts were transduced with the recombinant adenovirus. Treatment of the fibroblasts with CGH did not result in an increase in reporter gene induction. In another experiment, human primary adipocytes were also transduced with the recombinant adenovirus containing a reporter construct. Treatment of the human adipocytes with isoproterenol produced a 17-fold induction of luciferase expression. Treatment of the human adipocytes with CGH resulted in a 14-fold induction of the reporter gene. These results demonstrate CGH signaling through a GPCR in murine adipocytes and human adipocytes, and the production of cAMP levels similar to those achieved through β-adrenoreceptor stimulation.

Experimental Procedure

3T3 L1 cells were obtained from the ATCC (CL-173) and cultured in growth medium as follows: the cells were propagated in DMEM high glucose (Life Technologies, cat. # 11965-092) containing 10% bovine calf serum (JRH Biosciences, cat. # 12133-78P). Cells were cultured at 37° C. in an 8% CO₂ humidified incubator. Cells were seeded to collagen-coated 96-well plates (Becton Dickinson, cat. # 356407) at a density of 5,000 cells per well. Two days later, differentiation medium was added as follows: DMEM high glucose containing 10% fetal bovine serum (Hyclone, cat. # SH30071), 1 μg/ml insulin, 1 μM dexamethasone, and 0.5 mM 3-isobutyl-methyl xanthine (ICN, cat. #195262). The cells were incubated at 37° C. in 8% CO₂ for 4 days and the medium replaced with DMEM high glucose containing 10% fetal bovine serum and 1 μg/ml insulin. The cells were incubated at 37° C. in 8% CO₂ for 3 days, then the medium was replaced with DMEM high glucose containing 10% fetal bovine serum. The cells were incubated at 37° C. in 8% CO₂ for 3 days, and the medium was replaced with DMEM low glucose (Life Technologies, cat. # 12387-015) containing 10% fetal bovine serum. The day before the assay, the cells were rinsed with F12 Ham (Life Technologies, cat. # 12396-016) containing 2 mM L-glutamine (Life Technologies, cat. # 25030-149), 0.5% bovine albumin fraction V (Life Technologies, cat. # 15260-037), 1 mM MEM sodium pyruvate (Life Technologies, cat. # 11360-070), and 20 mM HEPES. Cells were transduced with AV KZ55, an adenovirus vector containing KZ55, a CRE-driven luciferase reporter cassette, at 5,000 particles per cell. Following overnight incubation, the cells were rinsed once with assay medium (F12 HAM containing 0.5% bovine albumin fraction V, 2 mM L-glutamine, 1 mM sodium pyruvate, and 20 mM HEPES). 50 μl of assay medium were added to each well followed by 50 μl of 2× concentrated test protein. The plate was incubated at 37° C. at 5% CO₂ for 4 hours. Medium was removed from the plate and the cells were lysed with 25 μl per well of 1× cell culture lysis reagent supplied in a luciferase assay kit (Promega, cat. # E4530). The cells were incubated at room temperature for 15 minutes. Luciferase activity was measured on a microplate luminometer (PerkinElmer Life Sciences, Inc., model LB 96V2R) following automated injection of 40 μl of luciferase assay substrate into each well. The method described above, with modifications, was also used to test CGH and isoproterenol on human adipocytes obtained from Stratagene (cat. # 937236) seeded in 96-well plates. Human adipocytes were rinsed once with basal medium (Stratagene, cat. # 220002) containing 0.5% bovine albumin fraction V, then transduced with AV KZ55 at 5,000 particles per cell. Following overnight incubation, the cells were rinsed once with assay medium comprised of basal medium containing 0.5% bovine albumin fraction V and assayed as described above.

### EXAMPLE 2

### CGH-Induced Lipolysis in 3T3 L1 Adipocytes

Summary

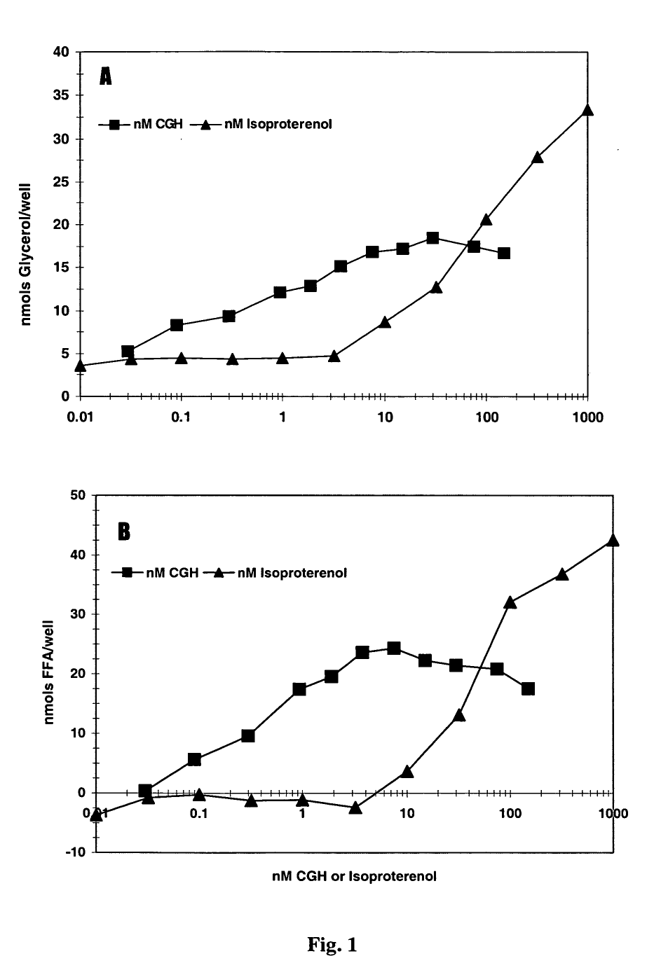

3T3 L1 Adipocytes were treated with CGH and the non-specific β-adrenoreceptor agonist isoproterenol for 4 hours. Lipolysis was assessed by the accumulation of glycerol and FFAs in the conditioned medium. FIG. 1 displays dose-response curves of CGH and isoproterenol for glycerol (panel A) and FFA (panel B). CGH potently stimulated lipolysis in the murine adipocytes, as shown in FIG. 1.

Measurement of Free Fatty Acids in Conditioned Media from Differentiated 3T3 L1 Cells

Free fatty acids were measured using the Wako NEFA C kit for quantitative determination of non-esterified (or free) fatty acids with a modified protocol. Isoproterenol (ICN), a lipolysis-inducing positive control, was diluted to a starting concentration of 2 μM in assay medium (Life Technologies low glucose DMEM, 1 mM sodium pyruvate, 2 mM L-glutamine, 20 mM HEPES, and 0.5% BSA). The isoproterenol was further diluted in half log serial dilutions. CGH was serially diluted down to 0.06 nM. Medium was removed from 3T3 L1 adipocytes in 96-well plates. 50 μl of assay medium were added to each well, followed by 50 μl of CGH or isoproterenol to each well. The plates were incubated for 4 hours at 37 degrees. 40 μl of conditioned medium were collected for glycerol assay analysis, and 40 μl of conditioned medium were collected for free fatty acid analysis. Oleic acid (Sigma) was dissolved in methanol and used as a reference for determining the amount of free fatty acids in the conditioned media. Wako reagents A and B were reconstituted to 4× the recommended concentration. Conditioned media samples were assayed in 96-well plates. 50 μl of Wako reagent A were added to 5 μl of oleic acid standard plus 40 μl of assay medium. 50 μl of Wako reagent A were added to 40 μl of conditioned medium from differentiated 3T3 L1 cells and 5 μl of methanol. The 96-well plates were incubated at 37° C. for 10 minutes. 100 μl of Wako reagent B were added to each well. The 96-well plates were incubated at 37 degrees for 10 minutes. The 96-well plates were then allowed to sit at room temperature for 5 minutes. The 96-well plates were centrifuged in a Beckman Coulter Allegra 6R centrifuge at 3250×g for 5 minutes to remove air bubbles. The absorbance at 530 nm was measured on the Wallac Victor2 Multilabel counter.

Measurement of Glycerol in Conditioned Media From Differentiated 3T3 L1 Cells

Glycerol was measured in conditioned media using the Sigma Triglyceride (GPO-Trinder) kit with a modified protocol. Isoproterenol was diluted to a starting concentration of 2 μM. The isoproterenol was further diluted in half log serial dilutions. CGH was diluted to starting concentrations of 300 nM in assay medium. CGH was then serially diluted down to 0.06 nM. Medium was removed from 3T3 L1 adipocytes in 96-well plates. 50 μl of assay medium were added to each well, followed by 50 μl of CGH or isoproterenol to each well. The plates were incubated for 4 hours at 37 degrees. 40 μl of conditioned medium were collected for glycerol assay analysis, and 40 μl of conditioned medium were collected for free fatty acid analysis. The glycerol standard was diluted in water to a range from 200 nmols/10 μl to 0.25 nmols/10 μl. Glycerol was used as a reference for determining the amount of glycerol in the conditioned media. Sigma reagent A was reconstituted to the recommended concentration. Conditioned media samples were assayed in 96-well plates. 150 μl of Sigma reagent A were added to 10 μl of glycerol standard plus 40 μl of assay medium. 150 μl of Sigma reagent A were added to 40 μl of conditioned medium from differentiated 3T3 L1 cells plus 10 μl of water. The 96-well plates were incubated for 15 minutes at room temperature. The 96-well plates were centrifuged in a Beckman Coulter Allegra 6R centrifuge at 3250×g for 5 minutes to remove air bubbles. The absorbance at 530 nm was measured on the Wallac Victor2 Multilabel counter.

### EXAMPLE 3

### Stimulation of Lipolysis by CGH in Vivo

Summary

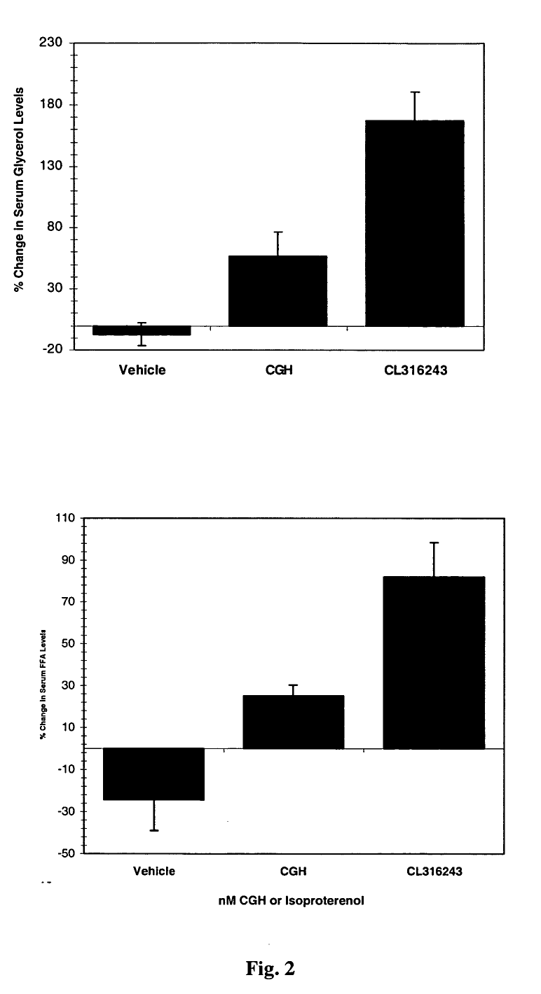

CGH, the β₃-adrenoreceptor agonist CL 316,243 (CL), and saline vehicle were examined for stimulation of lipolysis in mice following an overnight fast. Mice (n=4) were bled immediately before EP injection of CGH (300 μg/kg), CL (1 mg/kg), or vehicle, and then sacrificed 2 hours later. Lipolysis was assessed as the percent change in serum glycerol or FFA over the 2 hour period. FIG. 2 shows the changes in glycerol (upper panel) and FFA (lower panel) for the treatment groups. The serum glycerol and FFA for the vehicle groups decreased by 7%+/−9% and 24%+/−15%, respectively. The serum glycerol for the CGH group increased by 57%+/−20%; p=0.0254, and the FFA levels increased 25%+/−5%; p=0.0188. The serum glycerol for the CL group increased 168%+/−23%; p=0.0004, and the FFA increased 82%+/−16%; p=0.0029.

Treatment Protocol

C57 BL/6 male mice, age 19 weeks, were grouped to normalize weight (n=4 for each treatment; average group weight=37.8 g+/−0.4 g). Mice were housed individually for 18 hours prior to treatment, at which time food was withdrawn, with free access to water given. At approximately 8 a.m., the subjects were anesthetized with halothane and blood samples taken by retro-orbital eye bleed. The blood was allowed to clot, and the serum was separated by centrifugation and frozen for later analysis. Test substances were administered by IP injection in a volume of 0.1 ml, and the animals replaced in their cages for 2 hours with free access to water. At 2 hours, the mice were sacrificed and blood drawn by cardiac puncture.

Measurement of Glycerol and FFA in Murine Serum

For measuring free fatty acids in serum, the method previously described for measuring free fatty acids in conditioned media was followed, with the following modifications. Wako reagents A and B were reconstituted to 2× the recommended concentration. 75 μl of Wako reagent A were added to 5 μl of oleic acid standard plus 5 μl of water. 75 μl of Wako reagent A were added to 5 μl of serum plus 5 μl of methanol (to mirror the oleic acid standard conditions). The 96-well plates were incubated at 37degrees for 10 minutes. 150 μl of Wako reagent B were added to each well. The 96-well plates were incubated at 37° C. for 10 minutes. The 96-well plates were allowed to sit at room temperature for 5 minutes. The 96-well plates were centrifuged in a Beckman Coulter Allegra 6R centrifuge at 3250×g for 5 minutes to remove air bubbles. The absorbance at 530 nm was measured on the Wallac Victor2 Multilabel counter. For measuring glycerol in serum, the method previously described for measuring glycerol in conditioned media was followed, with the modifications described below. Sigma reagent A was reconstituted to 0.5× the recommended concentration. 200 μl of Sigma reagent A were added to 10 μl of glycerol standard. 200 μl of Sigma reagent A were added to 5 μl of serum plus 5 μl of water. The 96-well plates were incubated for 15 minutes at room temperature. The 96-well plates were centrifuged in a Beckman Coulter Allegra 6R centrifuge at 3250×g for 5 minutes to remove air bubbles. The absorbance at 530 nm was measured on the Wallac Victor2 Multilabel counter.

### EXAMPLE 4

### Expression and Purification of Recombinant CGH

Summary

A Chinese Hamster Ovary (CHO) cell line overexpressing both GPHA2 and GPHB5, the subunits of CGH, was generated and named CHO 180. CHO 180 was found to secrete active, heterodimeric CGH. CGH was purified from the supernatant of CHO 180 using standard biochemical techniques.

Generation of CHO 180

The CGH-producing cell line CHO 180 was generated in two stages. A construct expressing GPHA2, GPHB5 and drug resistance (dihydrofolate reductase) from the CMV promoter was transfected to protein-free CHO DG44 cells (PF CHO) by electroporation. The resulting pool was selected and amplified using methotrexate. Early analysis indicated a high level of GPHA2 expression, but a low level of GPHB5 expression. Therefore, a second construct expressing GPHB5 from the CMV promoter and zeocin resistance from the SV-40 promoter was transfected into the selected, amplified pool by electroporation. After zeocin selection, the final pool (CHO 180) expressed significant levels of both GPHA2 and GPHB5; the proteins were secreted as the non-covalent heterodimer, CGH.

Purification of CGH From CHO Culture Supernatant

CGH was purified from CHO culture supernatant by established chromatographic procedures: first the CGH was captured on a strong cation exchanger, POROS HS50; next it was affinity purified using ConA Sepharose; and finally was polished and buffer-exchanged into PBS by Superdex 75 size exclusion chromatography.

Cation Exchange Chromatography

The CHO culture supernatant was 0.2 μm filtered and adjusted to pH 6 and 20 mM 2-Morpholinoethanesulfonic Acid (MES). The CGH in the adjusted supernatant was captured at 55 cm/hr using a 1:2 online dilution with 20 mM MES pH 6 onto a POROS HS 50 column that was previously equilibrated in 20 mM MES pH 6. After loading was complete, the column was washed with 20 column volumes (CV) of equilibration buffer. This was followed by a 3 CV wash with 250 mM NaCl in 20 mM MES pH 6 at 90 cm/hr. Next the CGH was eluted from the column with 3 CV of 500 mM NaCl in 20 mM MES pH 6 at the same flow rate. Finally the column was stripped with steps of 1M and 2M NaCl and then re-equilibrated with 20 mM MES pH 6. The 500 mM NaCl-eluted pool containing the CGH was adjusted with NaOH to pH 7.4 for the next step.

ConA Sepharose Chromatography

ConA Sepharose is Concanavalin A coupled to Sepharose. Concanavalin A is a lectin, which binds reversibly to molecules, which contain D-mannopyranosyl, D-glucopyranosyl and related residues. The adjusted pool of CGH from the cation exchange chromatography was applied directly at 2 cm/hr to the ConA column equilibrated in 20 mM Tris pH 7.4 containing 0.5 M NaCl. After loading, the column was washed with 20 CV of equilibration buffer. The CGH was then competed off the column at 1-2 cm/hr with 3 CV of 0.5M Methyl-D-Manno-Pyranoside in 20 mM Tris pH 7.4. This CGH pool was concentrated via ultrafiltration using an Amicon stirred cell with a 5 kDa-cutoff membrane.

Size-Exclusion Chromatography

The concentrated CGH ConA pool was then applied to an appropriately sized bed of Superdex 75 resin (i.e. ≦5% of bed volume) for removal of remaining HMW contaminants and for buffer exchange into PBS. The CGH eluted from the Superdex 75 column at about 0.65 to 0.7 CV and was concentrated for storage at −80 ° C. using the Amicon stirred cell with a 5 kDa-cutoff ultrafiltration membrane. The heterodimeric protein was pure by Coomassie-stained SDS PAGE, had the correct NH2 termini, the correct amino acid composition, and the correct mass by SEC MALS. The overall process recovery estimated by RP HPLC assay was 50-60%.

We claim:

1. A method for inducing lipolysis in an individual comprising administering a pharmaceutically effective amount of corticotroph-derived glycoprotein hormone (CGH) to said individual, wherein CGH is a heterodimeric protein comprised of the polypeptides of SEQ ID NO:3 and SEQ ID NO:6.

2. A method for inducing weight loss in an individual comprising administering a pharmaceutically effective amount of CGH to said individual.

3. A method for treating type-2 diabetes in an individual comprising administering a pharmaceutically effective amount of CGH to said individual.

4. A method for improving insulin sensitivity in an individual comprising administering a pharmaceutically effective amount of CGH to said individual.

5. The method of claim 4 wherein said individual is obese.

6. The method of claim 5, wherein CGH is a heterodimeric protein comprising the polypeptides of SEQ ID NO:3 and SEQ ID NO:6.

7. The method of claim 1 wherein the method comprises activating adipocytes expressing the thyrotropin-stimulating hormone receptor (TSHR) comprising:
providing cells expressing the TSHR;
contacting the receptor with corticotroph-derived glycoprotein hormone (CGH),
wherein CGH is a heterodimeric protein comprising the polypeptides of SEQ ID NO:3 and SEQ ID NO:6; and
wherein the CGH heterodimer activates adipocytes.

8. The method according to claim 7 wherein the adipocyte activation is measured by an increase in cAMP production.

9. The method of claim 8 wherein the CGH heterodimer stimulates signal transduction of the adipocytes.

10. The method according to claim 9 wherein signal transduction is measured by an increase in cAMP production.


SEQUENCE LISTING (11 sequences)

SEQ ID NO: 1 - DNA, 746 bp, Homo sapiens
  CDS (56)...(442)
ccagcaggaggcacaggaaaactgcaagccgctctgttcctgggcctcggaagtgatgcctatggcgtcccctcaaaccctggtcctctatctgctggtcctggcagtcactgaagcctggggccaggaggcagtcatcccaggctgccacttgcaccccttcaatgtgacagtgcgaagtgaccgccaaggcacctgccagggctcccacgtggcacaggcctgtgtgggccactgtgagtccagcgccttcccttctcggtactctgtgctggtggccagtggttaccgacacaacatcacctccgtctctcagtgctgcaccatcagtggcctgaagaaggtcaaagtacagctgcagtgtgtggggagccggagggaggagctcgagatcttcacggccagggcctgccagtgtgacatgtgtcgcctctctcgctactagcccatcctctcccctccttcctcccctgggtcacagggcttgacattctggtgggggaaacctgtgttcaagattcaaaaactggaaggagctccagccctgatggttacttgctatggaatttttttaaataaggggagggttgttccagctttgatcctttgtaagattttgtgactgtcacctgagaagaggggagtttctgcttcttccctgcctctgcctggcccttctaaaccaatctttcatcattttacttccctctttgcccttacccctaaataaagcaagcagttcttga

SEQ ID NO: 2 - protein, 129 aa, Homo sapiens
MPMASPQTLVLYLLVLAVTEAWGQEAVIPGCHLHPFNVTVRSDRQGTCQGSHVAQACVGHCESSAFPSRYSVLVASGYRHNITSVSQCCTISGLKKVKVQLQCVGSRREELEIFTARACQCDMCRLSRY

SEQ ID NO: 3 - protein, 106 aa, Homo sapiens
QEAVIPGCHLHPFNVTVRSDRQGTCQGSHVAQACVGHCESSAFPSRYSVLVASGYRHNITSVSQCCTISGLKKVKVQLQCVGSRREELEIFTARACQCDMCRLSRY

SEQ ID NO: 4 - DNA, 390 bp, Homo sapiens
  CDS (1)...(390)
atgaagctggcattcctcttccttggccccatggccctcctccttctggctggctatggctgtgtcctcggtgcctccagtgggaacctgcgcacctttgtgggctgtgccgtgagggagtttactttcctggccaagaagccaggctgcaggggccttcggatcaccacggatgcctgctggggtcgctgtgagacctgggagaaacccattctggaacccccctatattgaagcccatcatcgagtctgtacctacaacgagaccaaacaggtgactgtcaagctgcccaactgtgccccgggagtcgaccccttctacacctatcccgtggccatccgctgtgactgcggagcctgctccactgccaccacggagtgtgagaccatc

SEQ ID NO: 5 - protein, 130 aa, Homo sapiens
MKLAFLFLGPMALLLLAGYGCVLGASSGNLRTFVGCAVREFTFLAKKPGCRGLRITTDACWGRCETWEKPILEPPYIEAHHRVCTYNETKQVTVKLPNCAPGVDPFYTYPVAIRCDCGACSTATTECETI

SEQ ID NO: 6 - protein, 106 aa, Homo sapiens
ASSGNLRTFVGCAVREFTFLAKKPGCRGLRITTDACWGRCETWEKPILEPPYIEAHHRVCTYNETKQVTVKLPNCAPGVDPFYTYPVAIRCDCGACSTATTECETI

SEQ ID NO: 7 - DNA, 5605 bp, Homo sapiens
atgaagctggcattcctcttccttggccccatggccctcctccttctggctggctatggctgtgtcctcggtgcctccagtgggaacctgcgcacctttgtgggctgtgccgtgagggagtttactttcctggccaagaagccaggctgcaggggccttcggatcaccacggatgcctgctggggtcgctgtgagacctgggaggtgagttgctaagttgtgcagatgacagtgtcttctaggccagcagcttgggtctgattcttaagagttcactttttaaatgatatgaggtagagctgggacatctgccctttcctgtggacttaaaaaaccaaaacaaaactatgattggcatcttccaaaagtgatttgaaaaacatgatgttgcccctctaacaaagcattgataaggttaagaatttggtttacattgtgtctatgtatctgggaatcatctctgggaggtcaagatgtactgttctacccgttttacagatgacatggagggattcaagggagagtggctgcaaagtcacgtagagcgtcagtgtaaagctgggaatcaatttgtggttcaagcttgtgacccaaactcctccctatgtttcctcattttggataaattagccagtttccaagaaagaggccctgagctgaagggtgagcgttggtcccagtgaagggtgagaccccttcactgcctcttctgcagcccttttcctcctcaagtctctgggagccctctggggttatcactgacggatccattaagttccttcatattcaattatacctggcctttttagagacatttaatttaaagtggagataacactctcaaacaaagttaaaatcctattgggctaagaggagctgtttgagtgatgaagaggaagagagctattcagcaccccagcagatcacattacgtagtgactgtgggctcttccccctgaggcctgcccacttggtaaccaatgaagtgctgtctctgatcttgtcactccctggcccaaaaaccttgaatgtccacacactactacagattcaataactaactttcaaggtgctcagcaatatggcgtctgcctgctttcctggagacagcacattttcttactctggccttggtaagtgactttcaaaggttttatcaaatagcccttatggatctcattttgttccttccctcatatcccttctccttcccatctgtcattatcatatttattcctgatgcctatctgcagtgccagctccctttctgggccttttttgacttgcaggtaagcccttgactatgctctacttttcgtcttacttcctcccccaccacacgcgtgatttaaattttttcaggacagaggttcattcttataaccttcacagcttttgtcaagatgtcgtgtatgaacaaggcattcaatacacatttgttggttgactgggatggacctccccctggagctgtagatcctccagcctaatggaaggccatttagaatcacacttgcactgtgagtggacactgccattgggaaaaatagccttctctttggggacccagagggtaacctgctcttgcttaggtacaattacggccctgtgaatggaattgggtcatagtgatgaaatctccaaattggatgaaactactctatcaaagtagttttcttttgcctcattcaggggcttgagccctactagcccaatgaaaatcgggttttgctaagtagactttgcctgtcaattggcagcaaattcacctggggcacttggcacctcctcctgttcagggactggcctggcagggcctctccctgttcgcatctagtgtctgggctatttgaagccctctctgtgccaaatcctcaaactcctgcttccgttcgattcagcccatcttctcttctttttaaaaactgatgaatgtctttaattggatcatggtcacccataggaggtcaggaactgtgctctcactggaaagatggaaacaccaaaaccgttaaagaacaagattctccctgatgttagccagctttcattcatgtcttgactgtgttatgaaaagggaggttacctatagaaaataaataaaagaatgagattcattttcccagcaatctgaaagtttctgcgctataaagcacttgattttttggtgggggggatcttaactgaaagcatgtctgaaaataaggatgttcatgatgacaggctggctggatttacatttgaaggttgttgaaaatagctattcctcataatctgggtatagagttgccagatttagcaaacaaacaaacagacaaacaaaataaaacaaaaccaatcccctccccacagaaacccaaactgaaataaaaccagaaaaccaggaagcccaggtaaatttgaatttaagataaataataaataaatttttagcataagtctgtctgtctcatacagtatttgggatgacttatactaaaaaattatgtatctgaaaatgaaattttatggggcgtttggtctgcctaggttcccagagtactaatggtaagaggacttaaagcaaatacgggaaggtaggagaaaacagttgaggacaaattcagctcttctggtctttgtcaaaggcaaggctggccgggcgtggtggctaacacctgtaatctcagcactttgggaggctgtggtgggtggataatgaggtcaggagttcgagaccagcctggccagtttttagtaaagaggtgagtaaaaccctgtctctactaaaaatacaaaaattagccgggcatggtggtatgcacctgtagtcccagctacttgggaggctgaggcagaagacttgcttgaacccaggaggtggaggttacagtgagccaagatcatgccactatactccagcctggcgacagagtgagactccatctcaaaaaaaaaaaaaaaaagaaaaaagaaaaaaaaaaggtaaggctgctattttcatgacattcatgcaagaacatcttgagttacatatgtatatatattcttttttgcctagaacaaagaagaaccaaaaagcaaaggtactgtcatttgaaagcttgttattatttacattactttcttataataattgcactaataagaacaatggattggctgggcgtggtggctcacgcctgtaatcccagcactttgggaggccgaggcaggcagatcacgaggtcaggaaatcgagaccatcctggctaacatggtgaaaccctgtctctactaaaaatacaaaaaatgagccaggcgtggtggtgggtgcctgtagtcccgggaggctgaggcaggagaatggcgtgaacccgggaggcggagattgcaatgagctgagattgcgccactgaactccagcctgggagacagcaagactccgtctcaaaaaaaaaaaaaaatggattgcattttttgaacatttactttgttctagacattgtgcattgcgtatatcatcttaccttatctctcaaacaatggtgggaggtagctattttgttttacagaggaggaaacttgagtcttcaggaagttaagtggattttccaaggtctccagcaagtggcagaacagggactcaagctccttagttctgactgcagggctcgagattttaactccagctaggtgctgatattttttctgatctgtgtgttctgtttatcaaaattgtctttgaacttaagatttataaaaggtgaaggaaggaaatgaatctttttgatgatcagaacagtgcacagagtattcgggaacctgtcttgtaatgttttctttcattgattcaatgacaaatagttattgaaactctcccagggtctgttttgggtacttgaggcacagtgggcaaaaatctctgtcctaaaagagcttactttctagagtgggaggaatatcacacgaatgaaaggtagactacgtcgtgtggtattgatcagtgctgtggtggaaaataaagcaagatgggggatgggaagtttctgggcatggagatggaatgttgcaattttaaataggatggtcaggaaatgcttccctgagagggtgacattctaacaaaaacccaaggttggtgaaagagtgaatcatacgggagaagaatgttccaggcagaaggaacagtaagtgcaaaggccctgagctggggctgttcctggtgggtcagaggagcaataaggagaccgccgtgagcctagtgaggaagtcagtgaggtgggaatggttgcaggcatttcagaaggtagagttgcagagaaggtgatgtaggtcttgaaggtgatcataaggtctttgatgtttgttctgagtgagatgggaaatcactggggctttgggcagaggagtaacatgatctgacttaggtttaaacaggatcactcagggccgctgtgttgcaaatagattgtagggagtaaaaatggaagaggggagaccagttagaaggtatttgcaatgactaagatgattcatttgctgactatgcatggagcacttgctgtgtgctatggtctctcctgggagcttagaatatggtcttgagtgaaatcagcttcttgctttcaggagtttgttttctactgggagacgacagagcaacaagtaaatcaacgaataacaagttaatttctgatagtgataaatgatactaaaaaactgaaacaagatcatatgttctaatgaattctctgtttctatctatggggacagaaacccattctggaacccccctatattgaagcccatcatcgagtctgtacctacaacgagaccaaacaggtgactgtcaagctgcccaactgtgccccgggagtcgaccccttctacacctatcccgtggccatccgctgtgactgcggagcctgctccactgccaccacggagtgtgagaccatctgaggccgctagctgctctctgcagacccacctgtgtgagcagcacatgcagttatacttcctggatgcaagactgtttaatttcgaccacacccatggaggaggttacctgtcgccccttaggtccagctcaggcaaaaggcccaaatgcagcctacttatgctaaaagttcaaaacaatattcgtgccttcaccaaaataatttctccagctcacatacctgcaaattaatttttctttgccttgagtcttggaacataatttgtgtatcacaatcctcccccaatttggacttataatatgctaatgatttaaacacatgggatgtaattaggatatggggctggaaagtctttaaattctcatgttctatttaacctctgatctccaaccggatttatgattaaagggctagaaatgaacaaaacccatgtactagtcttccttaccccagaggaattccagctgcaagcttctttagggaaaatgctcccttccccttttaactgagcaattatctacacaagaaataagactgctcagatatacaaagagagtagcttcaatgaaaagatgtttggatttggataattcttttccctagcaaaattcgctagctcccttaagagtcttaataaagaggctacgttgggattaaaagaaaaaaaaacagaaataaaatat

SEQ ID NO: 8 - DNA, 22 bp, Homo sapiens
tcagaagaaaatcagaggaatc

SEQ ID NO: 9 - DNA, 23 bp, Homo sapiens
gggacgttcagtagcggttgtag

SEQ ID NO: 10 - DNA, 20 bp, Homo sapiens
ctgcccatggacaccgagac

SEQ ID NO: 11 - DNA, 23 bp, Homo sapiens
ccgtttgcatatactcttctgag